(19) United States
(12) Patent Application Publication
(10) Pub. No.: US 2007/0062410 A1
(43) Pub. Date: Mar. 22, 2007

(54) Coloring liquid for the homogeneous coloring of ceramic materials

(76) Inventors: Norbert Thiel, Bad Sackingen (DE); Marc Stephan, Lorrach (DE)

Correspondence Address: JACOBSON HOLMAN, 400 SEVENTH STREET, N.W., WASHINGTON, DC, 20004, US

(21) Appl. No.: 11/231,292

(22) Filed: Sep. 21, 2005

(51) Int. Cl. C09D 11/00; C04B 14/00; A61L 33/00; B05D 3/00

(52) U.S. Cl. 106/31.05; 106/401; 106/400; 106/453; 106/456; 106/479; 106/499; 106/501.1; 427/2.1

(57) ABSTRACT

A coloring liquid which contains metal salts as a coloring component for the homogeneous coloring of ceramic workpieces made of oxide ceramics, the coloring liquid having a viscosity of from 10 cP (centipoise) to 100 cP, preferably from 30 cP to 60 cP (as measured with a Brookfield Rheometer DV III and RV spindle according to DIN 53018).

The present invention relates to a metal salt solution for the coloring of oxide ceramics, especially dental ceramics. The coloring solution is characterized by a viscosity which can be adjusted in accordance with the porosity of the oxide ceramics to be colored. It is suitable for coloring dental ceramics, especially those made of Y-TZP or Al₂O₃, which are in an open-pore presintered state prior to coloring.

DE-A-20 12 304 describes a method by which colored ceramic materials can be successfully prepared with maintaining the remaining operations, such as formulation and shaping methods, without staining the preparation device.

After formulation and shaping, the shaped bodies, which have been brought into a highly porous state by biscuit firing, are dipped into a solution of inorganic metal compounds. After soaking and drying, the bodies are fired as with non-colored ceramics, According to the invention, elements of Periodic Table groups 4 to 7, in particular, can be used in the solutions. In 1999, this method was transferred by some dental ceramics manufacturers to the coloring of preferably zirconia-based ceramics in colors similar to dentin.

Bioceramics 11, 138-141, describes the influence of metal oxides on the color of zirconium dioxide for dental application (see also FR-A-2 781 366). The metals examined by Cales and their color effect in sintered Y-TZP are listed in the following Table.

| Coloring Activity | Effective Color |
|---|---|
| Silver | Gray |
| Iron | Brownish |
| Zinc | Uncolored |
| Erbium | Light violet |
| Neodynium | Light pink |
| Cerium | Cream |
| Bismuth | Deep cream |
| Cerium | Orange |
| Terbium | Light orange |
| Manganese | Black |
| Praseodymium | Deep yellow |

The coloring of ceramics by means of aqueous metal complex solutions was also published in 1986 by Brigitte Klein and Adolf Nebelung [Klein, B., and Nebelung, A. (1986): “Färben von keramischen Gegenständen mittels wässriger MERAPON-Metallkomplex-Lösungen”, Sprechsaal 119: 922-924], which dealt with iron complexes, in particular. The article describes a method In which a metal complex solution is applied to the dried ceramic body by spraying, dipping or brushing, and the aqueous solution penetrates through the pores into the ceramic body. In the course of the subsequent firing process, a decomposition of the organic component occurs at first. When the temperature is increased further, the metal oxide formed reacts with the ceramic mass to result In a coloring of the ceramic body.

Iron, whose salt can be used in the form of chlorides, acetates or alcohols as well as oxo complexes, has proven particularly suitable for dental use (WO-A-00/46168).

However, a considerable drawback of the methods for use with dental restorations is the non-homogeneity of the coloring in surface and peripheral regions. Especially In peripheral regions, a supersaturation with color pigments tends to occur.

It is the object of the present invention to provide a coloring liquid with which the above mentioned drawbacks can be avoided.

This object is achieved by a coloring liquid which contains metal salts as coloring components for the homogeneous coloring of ceramic workpieces made of oxide ceramics. The coloring liquid according to-the invention has a viscosity of from 10 cP (centipoise) to 100 cP, preferably from 30 cP to 60 cP (as measured with a Brookfield Rheometer DV III and RV spindle according to DIN 53018).

The advantages of the invention include the fact that the viscosity of the coloring liquid can be adjusted with respect to the open-pore property of the oxide ceramics to be colored. This has the effect that the coloring liquid can completely infiltrate the porous workpiece, while a quick drying and replacement flowing of the liquid is avoided, The adjustment of the viscosity is preferably effected by carbohydrates, especially monosaccharides or disaccharides.

The coloring liquid according to the invention serves especially for the infiltration of presintered oxide-ceramic bodies having an open porosity with a density of from 20% to 90% of the theoretical density, preferably from 45% to 82% of the theoretical density.

In particular, the coloring liquid according to the invention contains soluble compounds of at least one of the metals of Periodic Table groups 6 to 7, especially iron, chromium and manganese.

Typically, the coloring liquid according to the invention is an aqueous-based solution.

In particular, chlorates, nitrates, acetates, citrates and hydrates or combinations thereof are employed as metal salts in the coloring liquid according to the invention.

The preferred form of the incorporated metal salts is citrate, which does not release any nocuous decomposition products in the thermal process, in contrast to metal halides (HX) and metal nitrates (NOₓ).

Another advantage of the use of ferric citrate according to the invention is its excellent biocompatibility. Iron citrate is used, for example, in food supplements.

In the coloring liquid according to the invention, the concentration of the coloring component, such as the ion of a metal salt, is typically within a range of from 0.002% to 2% by weight.

For adjusting the viscosity of the coloring liquid according to the invention, carbohydrates, for example, can be employed, especially monosaccharides or disaccharides. Various possibilities of viscosity adjustment are known to the skilled person. When the mentioned monosaccharides or disaccharides are used, the skilled person knows that an increase of the concentration of such additives usually contributes to an Increase in viscosity. The exact viscosities to be adjusted as desired can be established in routine experiments, for example, by performing different serial experiments with coloring liquids having different viscosities on specimens having different porosities. For example, if the pores are relatively small, a high viscosity can have the effect that the coloring liquid will not sufficiently penetrate into the pores. In this case, a transition from a higher viscosity to a lower viscosity would have to be made.

The invention also relates to the use of the coloring liquid according to the invention for coloring dental-ceramic restorations.

The invention further relates to a method for homogeneously coloring ceramic workpieces made of oxide ceramics using a coloring liquid containing metal salts, wherein the oxide ceramics are infiltrated with the coloring liquid according to the invention while in a presintered open-pore state.

This is preferably followed by a step of condensation sintering of the oxide ceramics infiltrated with the coloring liquid according to the invention to obtain a homogeneously colored dental ceramic material.

In particular, the method according to the invention serves for coloring a ceramic workpiece for a ceramic dental restoration. For example, a dentin-like color can be achieved by using the coloring liquid according to the invention.

The resulting dental restoration may also be veneered.

### EXAMPLE 1

Coloring solution for Y-TZP:

Brightness stage 1 of VITA System 3D-Master

| | | |
|---|---|---|
| Water | 100 | ml |
| Disaccharide | 100 | g |
| Ferric citrate | 3 | g |
| Coloring solution | ˜160 | ml |

The water is heated to 80° C., and the ferric citrate is dissolved therein within 30 minutes. Subsequently, the solution is cooled down to about 30° C., and the disaccharide is added and completely dissolved with stirring. After having cooled down to room temperature, the coloring solution is filled into a light-tight vessel and sealed hermetically.

Now, the coloring of a workpiece made of Y-TZP which is in a porous state can be effected, for example, by dipping the workpiece into the coloring solution. The geometries which are usual in the dental field are completely soaked with the liquid after about two minutes After the workpiece has been removed from the dipping bath, the excess liquid is blotted from the surface, followed by sintering the workpiece. After the sinter firing, the workpiece has a homogeneous color which corresponds to the first brightness stage of the color scale of the VITA System 3D-Master. Expressed in L*a*b* values (Datacolor SF 600);

| L* | a* | b* |
|---|---|---|
| 77.81 | −2.56 | 12.51 |

### EXAMPLE 2

Coloring solution for Y-TZP:

Brightness stage 4 of VITA System 3D-Master

| | | |
|---|---|---|
| Water | 100 | ml |
| Disaccharide | 100 | g |
| Ferric citrate | 10 | g |
| Coloring solution | ˜160 | ml |

The coloring solution is prepared as in Example 1.

After the sinter firing, the workpiece has a homogeneous color which corresponds to the fourth brightness stage of the color scale of the VITA System 3D-Master. Expressed in L*a*b* values (Datacolor SF 600):

| L* | a* | b* |
|---|---|---|
| 71.26 | 2.76 | 26.01 |

### EXAMPLE 3

Coloring solution for Al₂O₃:

Brightness stage 2 of VITA System 3D-Master

| | | |
|---|---|---|
| Water | 100 | ml |
| Disaccharide | 100 | g |
| Ferric citrate | 4 | g |
| Chromic acetate | 0.05 | g |
| Coloring solution | ˜160 | ml |

The water is heated to 80° C., and the ferric citrate is dissolved therein within 30 minutes. Subsequently, the chromic acetate is added and dissolved. Then, the solution is cooled down to about 30° C., and the disaccharide is added and completely dissolved with stirring. After having cooled down to room temperature, the coloring solution is filled into a light-tight vessel and sealed hermetically.

Now, the coloring of a workpiece made of Al₂O₃ which is in a porous state can be effected, for example, by dipping the workpiece into the coloring solution. The geometries which are usual in the dental field are completely soaked with the liquid after about two minutes, After the workpiece has been removed from the dipping bath, the excess liquid is blotted from the surface, followed by sintering the workpiece. After the sinter firing, the workpiece has a homogeneous color which corresponds to the first brightness stage of the color scale of the VITA System 3D-Master. Expressed in L*a*b* values (Datacolor SF 600):

| L* | a* | b* |
|---|---|---|
| 79.09 | −0.18 | 25.23 |

1. A coloring liquid which contains metal salts as a coloring component for the homogeneous coloring of ceramic workpieces made of oxide ceramics, the coloring liquid having a viscosity of from 10 cP (centipoise) to 100 cP, preferably from 30 cP to 60 cP (as measured with a Brookfield Rheometer DV III and RV spindle according to DIN 53018).

2. The coloring liquid according to claim 1 for the infiltration of presintered oxide-ceramic bodies having an open porosity with a density of from 20% to 90% of the theoretical density, preferably from 45% to 82% of the theoretical density.

3. The coloring liquid according to claim 1, wherein the viscosity of the liquid can be adjusted in accordance with the porosity of the ceramic body.

4. The coloring liquid according to claim 1, containing soluble compounds of at least one of the metals of Periodic Table groups 6 to 7, especially iron, chromium and manganese.

5. The coloring liquid according to claim 1 as an aqueous-based solution.

6. The coloring liquid according to claim 1, wherein said metals salts are chlorates, nitrates, acetates, citrates and hydrates or combinations thereof.

7. The coloring liquid according to claim 1, wherein the concentration of the coloring component, such as an ion of the metal salt, is within a range of from 0.002% to 2% by weight.

8. The coloring liquid according to claim 1, wherein carbohydrates, especially monosaccharides or disaccharides, are employed for adjusting the viscosity.

9. Use of the coloring liquid according to claim 1 for coloring dental-ceramic restorations.

10. A method for homogeneously coloring ceramic workpieces made of oxide ceramics using a coloring liquid containing metal salts, wherein the oxide ceramics are infiltrated with the coloring liquid according to claim 1 while in a presintered open-pore state.

11. The method according to claim 10, which is followed by a step of condensation sintering of the oxide ceramics infiltrated with the coloring liquid to obtain a homogeneously colored dental ceramic material.

12. The method according to claim 10, wherein said ceramic workpiece is a colored ceramic dental restoration.

13. The method according to claim 12, wherein the color of natural dentin is achieved by using the coloring liquid.

14. The method according to claim 12, whereupon the resulting dental restoration is veneered with a suitable ceramic material.
